(19) United States
(12) Patent Application Publication
(10) Pub. No.: US 2018/0325445 A1
(43) Pub. Date: Nov. 15, 2018

(54) Dilation and Measurement Apparatus and Methods for Promoting and Assessing Cervical Ripening During Induction of Labor

(71) Applicant: Northwestern University, Evanston, IL (US)

(72) Inventors: Robert J. McCarthy, Wheaton, IL (US); Feyce Peralta, Chicago, IL (US)

(21) Appl. No.: 15/775,320

(22) PCT Filed: Nov. 17, 2016
(86) PCT No.: PCT/US16/62441

§ 371 Date: May 10, 2018

Related U.S. Application Data
(60) Provisional application No. 62/256,547, filed on Nov. 17, 2015.

(51) Int. Cl. A61B 5/00; A61B 5/053; A61B 5/107; A61M 25/10

(52) U.S. Cl. CPC A61B 5/435; A61B 5/0535; A61B 5/1076; A61M 29/02; A61B 2562/043; A61M 25/1011; A61B 5/0538; A61M 25/1002

(57) ABSTRACT

The disclosure provides apparatus and methods of promoting and assessing cervical dilation during induction of labor including apparatus that utilizes impedance planimetry for assessment of cervical dilation during cervical ripening with balloon, prior to induction of labor.

### CROSS-REFERENCE TO RELATED APPLICATION

This application claims the benefit and priority of U.S. Provisional Patent Application Ser. No. 62/256,547, filed Nov. 17, 2015 and PCT Application No. PCT/US16/62441, filed Nov. 17, 2016 the disclosures of which are hereby incorporated herein by reference in its entireties.

### FIELD OF THE DISCLOSURE

This disclosure relates generally to preparation of pregnant women for induced labor, and more particularly to apparatus and methods of promoting and assessing cervical dilation during induction of labor.

### BACKGROUND

Induction of labor is the technique by which uterine contractions are promoted to achieve vaginal delivery of a fetus prior to the onset of spontaneous labor. The overall rate of induction of labor in the United States is close to 1 in 5 births. The cervix is a ring of muscular tissue that separates the uterus from the birth canal. The maternal cervix undergoes numerous changes in preparation for labor and birth. Prior to induction of labor, the cervix thins out and softens to allow cervical dilation and passage of the fetus. This process, commonly referred to as cervical ripening, occurs over a period of hours. It is common for the Bishop scoring system to be used to assess the readiness of the cervix for induction of labor. When the cervix is deemed unfavorable for induction of labor (simplified Bishop score ≤5) different methods are used, alone or in combination, to promote cervical ripening, uterine contractions, and delivery of a fetus prior to the onset of spontaneous labor. Medications or devices may be used to help soften the cervix, so that it will dilate or stretch for labor. For instance, Cervical Ripening with Balloon (CRIB) is a method of physical softening, thinning and mechanical cervical dilation in preparation for labor and birth.

CRIB placement by a medical professional may be accomplished utilizing well accepted methods, for example, via insertion of a 26 French bulb catheter beyond the internal opening of the uterus (the internal cervical orifice or cervical os, which separates the body of the uterus from the cervix), for mechanical cervical ripening. Under such a procedure, a 26 Fr/30 cc Foley catheter may be lubricated, grasped with forceps 3 cm from the tip and inserted using a smooth, twisting motion into the endocervix by direct visualization or blindly by locating the cervix with the examining fingers and guiding the catheter over the hand and fingers through the endocervix and into the potential space between the amniotic membrane and the lower uterine segment. The CRIB balloon catheter is then inflated with 80 mL of 0.9% normal saline and retracted, so that it rests on the internal cervical os. Slight traction is applied to the end of the catheter and the tubing is taped to the inner aspect of the patient's thigh. The catheter may be left in place for 6-12 hours, or until spontaneous expulsion or rupture of membranes occurs, whichever occurs first. The direct pressure of the catheter in the uterus causes stress in the lower uterine segment and may cause the local production of prostaglandins.

While the catheter may be left in place for 6-12 hours, one undesirable limitation of this practice is that it requires periodic digital examinations of the cervix using the Bishop's score to assess the balloon's location and cervical readiness for its removal. Thus, cervical ripening must be periodically subjectively assessed by a gloved hand of a physician, midwife, or trained professional. This process is uncomfortable for the patient and delays the start of interventions in the induction process. In addition, when the balloon is placed, the premise is that it will come out once the cervix has reached 4-5 cm dilation, however, the balloon could migrate out to the vagina and this might not be immediately recognized since only periodic assessments are done, leading to a patient staying in the labor and delivery suite potentially without interventions (e.g., oxytocin administration).

Notably, there are no continuous methods in current clinical practice to promote ripening while continuously quantitatively determining the diameter of the cervix or otherwise assessing cervical dilation and the degree of ripening and readiness for induction of labor.

### SUMMARY

The present disclosure provides apparatus and methods to promote ripening while performing continuous measurements of cervical dilation during pre-induction of labor, without further intervention. The innovative apparatus and methods disclosed herein utilize a balloon that induces cervical ripening, but also is used to continuously measure cervical dilation. Thus, the device utilizes a method to continuously measure the diameter of the cervical opening and has the advantages of a CRIB dilation device, while also providing the advantages of a device that measures the diameter of a lumen using impedance planimetry. Impedance planimetry has been used to measure the dimensions and function of hollow organs, lumens and sphincteric regions, but has not been used in continuous measurement of dilation during a process of increasing dilation, such as with a CRIB dilation device. The ability to promote cervical ripening while continuously assessing such ripening, in turn, will reduce the discomfort and delays that may be associated with the existing deployment of a CRIB dilation device and periodic manual digital examinations for cervical readiness. In addition to these significant benefits to patients, there are additional benefits to medical professionals who will be able to promote dilation and receive accurate information for timing deliveries, via continuous and objective collection of information regarding cervical shape and dilation, from a single device.

In a first aspect, the disclosure provides an apparatus for promoting and assessing dilation of a cervix during induction of labor. The apparatus includes a catheter having at least one lumen, at least one dilation balloon connected to and in fluid communication with the at least one lumen of the catheter, the at least one dilation balloon extending along and encircling a length of the catheter, and the at least one dilation balloon being configured to be filled with fluid to expand sufficiently to promote dilation of the cervix. The apparatus further including a plurality of sensors connected to the at least one lumen of the catheter and being spaced apart along the length of the catheter encircled by the at least one dilation balloon, and the plurality of sensors being configured to measure the diameter of the at least one dilation balloon along the length of the catheter encircled by the at least one dilation balloon.

In a second aspect, the disclosure provides a method of promoting and assessing dilation of a cervix with a catheter apparatus having at least one lumen connected to at least one dilation balloon that encircles and extends along a length of the catheter and a plurality of sensors being spaced apart and providing diameter measurements of the at least one dilation balloon along the length of the catheter encircled by the dilation balloon. The method includes positioning the at least one dilation balloon within the cervix, filling the at least one dilation balloon with a fluid to expand sufficiently to promote dilation of the cervix, using the sensors to measure the diameter of the at least one dilation balloon along the length of the catheter encircled by the at least one dilation balloon, and periodically assessing the measurements of the diameter of the at least one dilation balloon along the length of the catheter encircled by the at least one dilation balloon to assess dilation of the cervix.

In a third aspect, the disclosure provides a method of promoting and assessing dilation of a cervix with a catheter apparatus that is connected to an ambulatory system. An ambulatory system may include a sensor/controller for the dilation and measurement catheter apparatus that is attached to the patient. Attachment may be achieved by a removable strap or other suitable means to hold the controller in place. The sensor/controller may be controlled directly by the patient by input to a screen, keypad or other interface provided on the sensor/controller. In another embodiment, the sensor/controller may be very small and connected wirelessly to a larger instrument having direct input controls for the patient to monitor and remotely control the sensor/controller. In another embodiment the sensor/controller of the ambulatory system on the patient, or the smaller sensor/controller may be controlled and report information to a mobile application that may both control the process and report the progress in real time, such as on a cellular device. This process and the software to control and report the process with this apparatus may be shared via the internet with a health care professional and/or provider and the patient, allowing the patient to be able to be mobile and, depending on the circumstances, to be dismissed to go home and to continue the process of CRIB and the measurements to assess progress toward readiness for labor.

It is to be understood that both the foregoing general description and the following detailed description are exemplary and provided for purposes of explanation only, and are not restrictive of the subject matter claimed. Further features and objects of the present disclosure will become more fully apparent in the following description of the preferred embodiments and from the appended claims.

### BRIEF DESCRIPTION OF THE DRAWINGS

In describing the preferred embodiments, references are made to the accompanying drawing figures, and wherein:

FIG. 1 shows a first example dilation and measurement apparatus.

FIG. 2 shows a close up view of the balloon portions of the first example apparatus.

FIG. 3 shows a close up view of the balloon portion of a second example dilation and measurement apparatus.

FIG. 4 shows a graphical representation of the progress of dilation as measured over a period of time for two different patients.

FIG. 5 shows an image of the starting measurements taken along the length of a balloon that corresponds to the Patient 9 data shown in FIG. 4.

FIG. 6 shows an image of the ending measurements taken along the length of a balloon that corresponds to the Patient 9 data shown in FIG. 4.

FIG. 7 shows a close up view of the balloon portions of a third example dilation and measurement apparatus.

FIG. 8 shows a close up view of a tubing interface for an example dilation and measurement apparatus.

FIG. 9 shows a view of the apparatus in a configuration with a fluid source bag positioned above a dilation and measurement balloon.

It should be understood that the drawings are not to scale. While some mechanical details of the example dilation and measurement apparatus and methods for promoting and assessing cervical ripening during induction of labor have not been shown, including other plan and section views of the particular components, such details are considered to be within the comprehension of those skilled in the art in light of the present disclosure. It also should be understood that the present disclosure and claims are not limited to the preferred embodiments illustrated.

### DETAILED DESCRIPTION

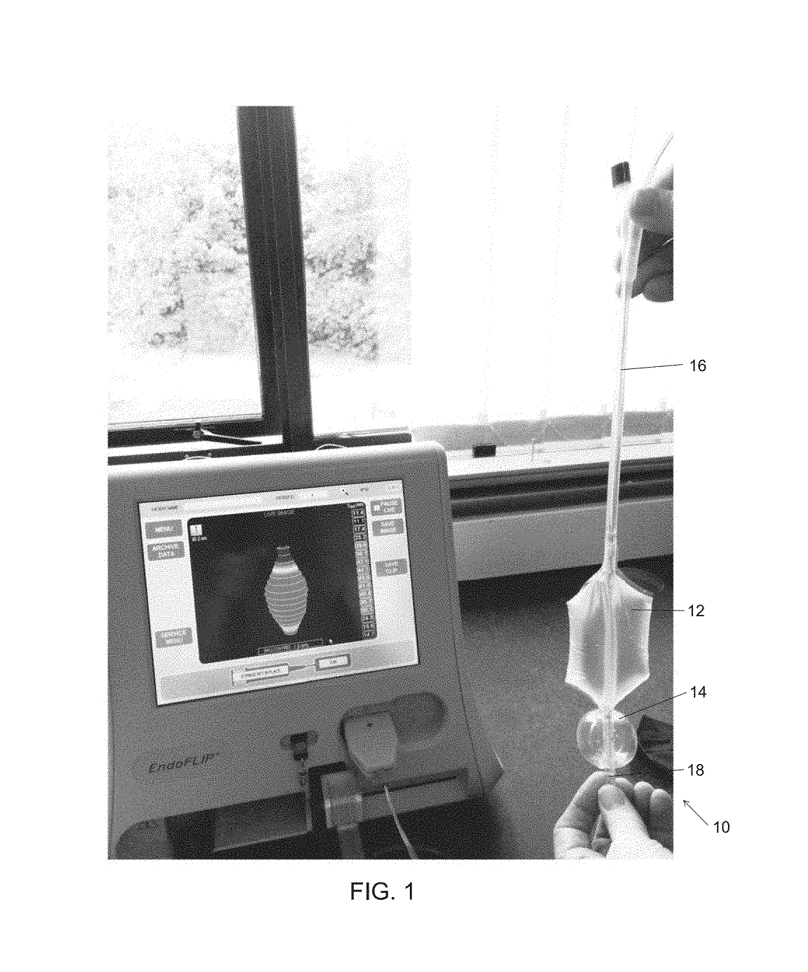

Referring generally to FIGS. 1-9, it will be appreciated that dilation and measurement apparatus and methods for promoting and assessing cervical ripening during induction of labor of the present disclosure generally may be embodied within numerous configurations.

FIG. 1 shows a first example dilation and measurement apparatus 10. The first example catheter apparatus 10 for dilation and measurement was formed by modifying a standard Foley catheter to have a first balloon 12 that is a dilation and measuring balloon having a size of approximately 5 cm×5 cm, along with a second smaller balloon 14. Thus, the first example apparatus includes a second smaller balloon 14 that encircles a catheter 16 near the distal end 18 of the catheter 16. The smaller balloon 14 may be helpful in locating and maintaining the position of the apparatus. As will be seen in the second example, use of this second smaller balloon 14 is optional and may be filled in a manner known in the art.

In the first example catheter apparatus 10, the larger first balloon 12 is positioned adjacent the proximal end of the second balloon 14 and extends along and encircles a length of the catheter 16. The larger first balloon 12 is used as a dilation balloon for promoting dilation, and advantageously also is used for measuring the progress of ripening. Accordingly, the balloon 12 may alternatively be referred to herein as a dilation balloon or as a dilation and measurement balloon. It will be appreciated that the catheter and balloons may be constructed of suitable materials commonly used in the industry, and may be dimensioned as desired. The dilation and measurement apparatus 10 additionally may be secured to the patient, such as by taping to a leg, to assist in maintaining the position during use.

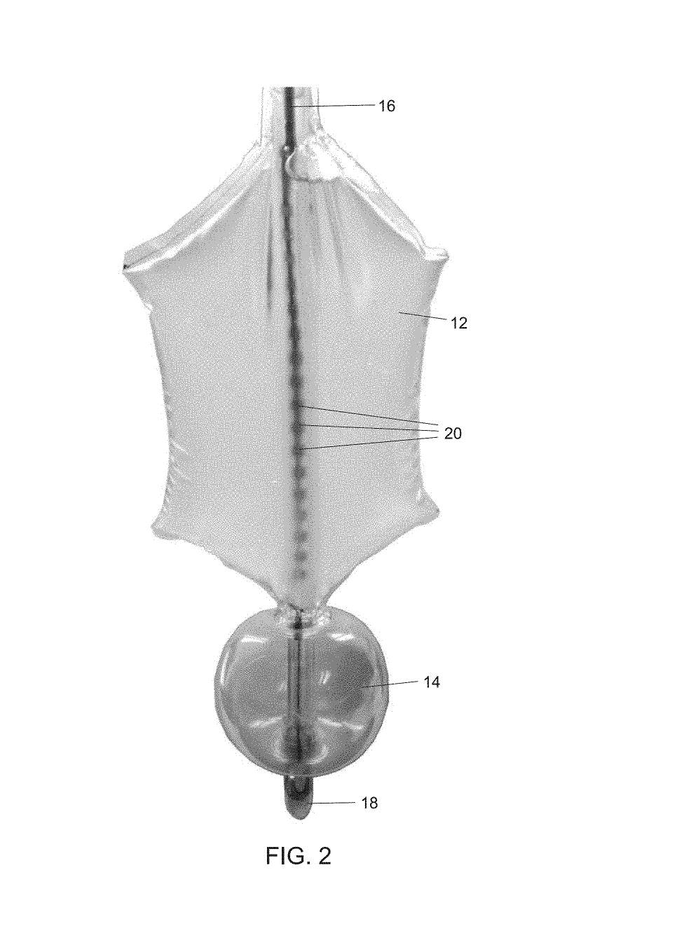

The modified Foley catheter 16 has two lumens, with one being employed for filling the smaller balloon 14, but the present apparatus and method do not use the second lumen of the modified Foley catheter for urine evacuation. Instead, the urine lumen is repurposed to allow measurement electrodes or sensors 20 to be introduced, as well as for injection of a conductive fluid into the larger dilation balloon 12 that also is used for measuring dilation. As better seen in FIG. 2 for this example, a plurality N of sensors 20 are spaced apart at 5 mm increments along the catheter and within the length of the dilation balloon 12. Three of the plurality of sensors 20 are identified in FIG. 2, for explanation purposes. The sensors 20 of the first example dilating and measurement balloon 12 are electrodes that measure voltage, and the sensors 20 are configured to interface with an endolumenal functional imaging probe, such as of an EndoFLIP® System from Crospon Inc., of Galway, Ireland. Such a system is shown in the background within FIG. 1.

Using the modified catheter 16 in the dilation and measurement apparatus 10, the system converts AC voltage measurements between pairs of sensors 20 to estimate diameter measurements radially outward from the sensors 20 using a principle known as impedance planimetry. Accordingly, the sensors 20 provide (N−1) estimated diameter measurements that correspond their positions along the length of the dilation balloon 12 that encircles the catheter 16. For instance, seventeen electrodes would provide sixteen estimated diameter measurements along the length of the dilation balloon.

It will be appreciated that the size of the dilation and measurement balloon and the number of electrodes or sensors and their position along the length of the catheter passing through the dilation and measurement balloon may be specified as desired. For instance, there may be dilation and measurement balloons of different dimensions for improved fit for patients of different sizes, and the electrodes or sensors need not necessarily be of a particular quantity or be spaced apart uniformly. This will be further demonstrated by the third example, which provides a dilation and measurement balloon that is shorter and includes fewer sensors with varied spacing.

The first example apparatus 10 may be used in a method of promoting and assessing dilation of a cervix. Thus, the method includes using a catheter apparatus 10 having at least one dilation balloon 12 that encircles and extends along a length of a catheter 16 and a plurality of sensors 20 being spaced apart and providing diameter measurements of the at least one dilation balloon 12 along the length of the catheter encircled by the dilation balloon 12. The method includes positioning the at least one dilation balloon 12 within the cervix, filling the at least one dilation balloon 12 with a fluid to expand sufficiently to promote dilation of the cervix, using the sensors 20 to measure the diameter of the at least one dilation balloon 12 along the length of the catheter encircled by the at least one dilation balloon 12, and periodically assessing the measurements of the diameter of the at least one dilation balloon 12 along the length of the catheter encircled by the at least one dilation balloon 12 to assess dilation of the cervix.

With this method, diluted saline, or another suitable solution, is injected or otherwise added to the dilation balloon 12 to achieve a pressure preferably between 30 and 50 mmHg. This pressure will allow fluid to distribute into the portion of the dilation and measurement balloon 12 located within the cervix as it starts to naturally expand during ripening. This pressure is not sufficient to actively dilate the cervix. However, the system advantageously may be configured to inject or otherwise add fluid to produce a greater pressure, such as 50 to 80 mmHg to additionally apply pressure to the cervix, so as to promote dilation. The system also advantageously may be configured to use the sensors 20 to provide periodic diameter measurement updates, using measurement information that may be gathered from the sensors, such as at a rate of 10 times per second, or as otherwise specified. The measurements and updates may be processed in a continuous manner or periodically, and over an extended period of time, such as several hours. This permits periodic assessment of the measurements and essentially alleviates the need for periodic manual digital examinations, thereby reducing the associated patient discomfort, increased risk of infection, and delays. This also provides more accurate and objective information to medical professionals, providing for real time assessment of cervical shape, dilation and readiness for labor.

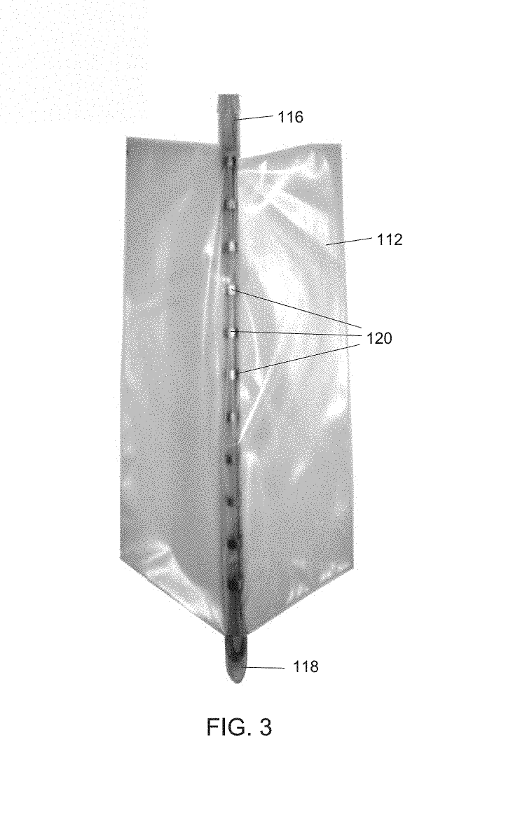

A close up view of a dilation and measurement balloon portion 112 of a second example dilation and measurement apparatus is shown in FIG. 3. The second example apparatus includes a single dilation balloon 112 located near the distal end 118 of a catheter 116, without the optional smaller balloon of the first example apparatus shown in FIGS. 1-2. The dilation balloon 112 of the second example has a configuration that is slightly more slender and shorter in length than the measurement and dilation balloon 12 of the first example. This, together with a bullet-shaped distal end 118 of the catheter 116, facilitate placement within the cervix of a patient. To further facilitate placement, a negative pressured dilation balloon may be utilized.

A plurality of eleven electrodes or sensors 120 are included along the catheter and within the length of the dilation balloon 112, which generate ten estimated diameter measurements that may be reported periodically and/or continuously, as desired. It will be appreciated that the single dilation balloon 112 of the second example catheter apparatus may be used for promoting dilation and measuring the progress of ripening, with the system providing the aforementioned advantages associated with continuous promotion and assessment of dilation and readiness for labor. The catheter 116 may be constructed with a single lumen for filling the dilation balloon 112 with fluid and for placement of the sensors 120. As noted with respect to the first example, the catheter 116 and dilation balloon 112 of the second example apparatus may be constructed of suitable materials commonly used in the industry and the apparatus additionally may be secured to the patient, such as by taping to a leg or by other suitable attachment methods. Thus, both examples utilize impedance planimetry for assessment of cervical dilation during cervical ripening with balloon (CRIB) prior to induction of labor.

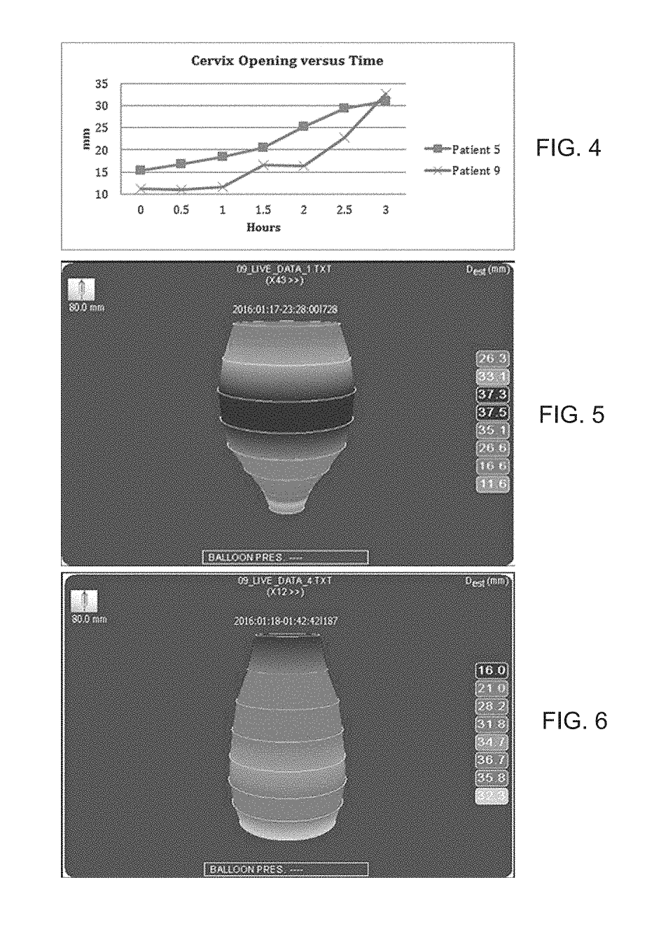

Turning to FIG. 4, a graphical representation is provided showing the result of measurements of the progress of dilation as measured over a period of time for two different patients. The starting and final measurements for the patients also correspond to further images shown respectively in FIGS. 5 and 6. Thus, measurements obtained by a dilation and measurement balloon having nine sensors that provided eight measurements of diameter along the length of the catheter encircled by the dilation balloon are shown at the start in FIG. 5, where the cervix measured 11.6 mm, and three hours later, as shown in FIG. 6, the cervix measured 32.3 mm. The progression in dilation for Patient 9 is represented graphically by the lower line in FIG. 4, and shows fairly steady progress. The respective level of dilation may be displayed in various ways visually, so as to provide immediate recognition of the progress of the dilation.

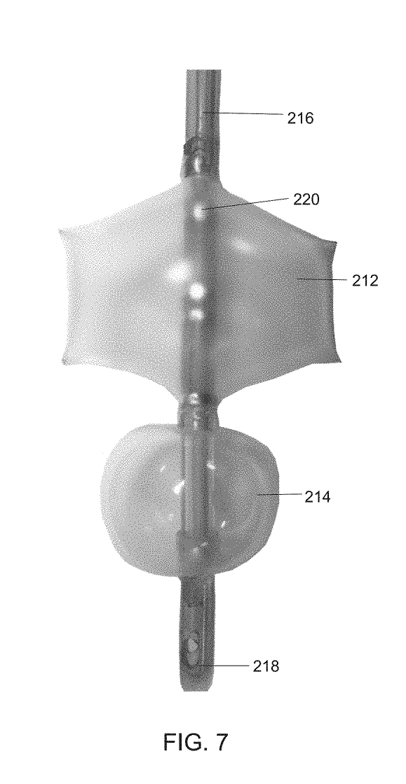

Turning to FIG. 7, a close up view is provided for the balloon portion of a third example dilation and measurement apparatus. The third example catheter apparatus for dilation and measurement includes a smaller balloon 214 that encircles the catheter 216 near the distal end 218 of the catheter. The balloon 214 may serve a similar purpose to that which was discussed for the smaller distal balloon of the first example apparatus. The third example dilation and measurement apparatus also includes a balloon 212 for dilation and measurement. The larger dilation balloon 212 is positioned near the proximal end of the smaller balloon 214 and extends along and encircles a length of the catheter 216. As in the first example, the larger dilation and measurement balloon 212 is used as a dilation balloon for promoting dilation, and advantageously also is used for measuring the progress of ripening.

The third example apparatus provides electrodes or sensors 220 for measuring the diameter of the expanded balloon along the portion of the catheter 216 that is surrounded by the dilation balloon 212. The third example apparatus includes far fewer sensors than the other examples, but it will be appreciated that the apparatus may still provide an adequate number of measurements, with the lowermost measurement likely providing the best representation of the progress of dilation and ripening in terms of readiness for labor.

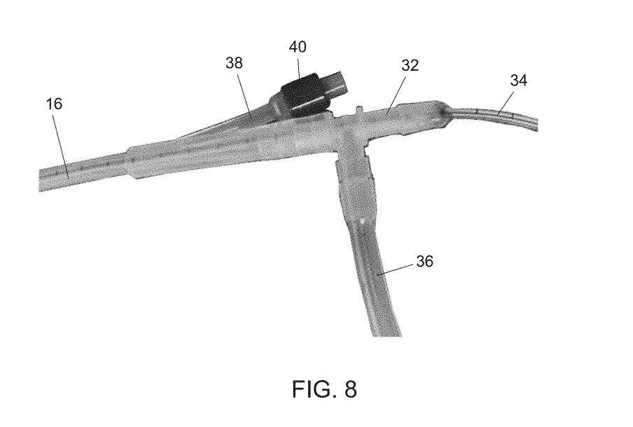

FIG. 8 shows a close up view of a tubing interface for an example dilation and measurement apparatus, such as the one shown in FIG. 1. The tubing interface 30 would be provided at a proximal end of the catheter 16. This example interface 30 also includes a tube 32 that receives the wires 34 that extend from the imaging equipment to the electrodes or sensors 20. The interface 30 further includes a tube 36 from a fluid source, and a tube 38 that may include a relief valve 40, by which pressure may be relieved or fluid drained from the dilation balloon.

While the fluid source that would be connected to the tube 36 and provide fluid to fill the dilation and measurement balloon 12 may include a syringe or a machine that is capable of metering out fluid, such devices typically would provide fluid on a volume basis, such as in millilitres (ml). While this method may be acceptable in many situations, it may have drawbacks in circumstances where a trapped volume of fluid is subjected to additional influences, such as when a patient might roll onto a side, which may increase the pressure to an unacceptable level. In such an event, the relief valve 40 may be utilized to prevent a spike in fluid pressure, for the safety of the patient and to protect the apparatus from damage.

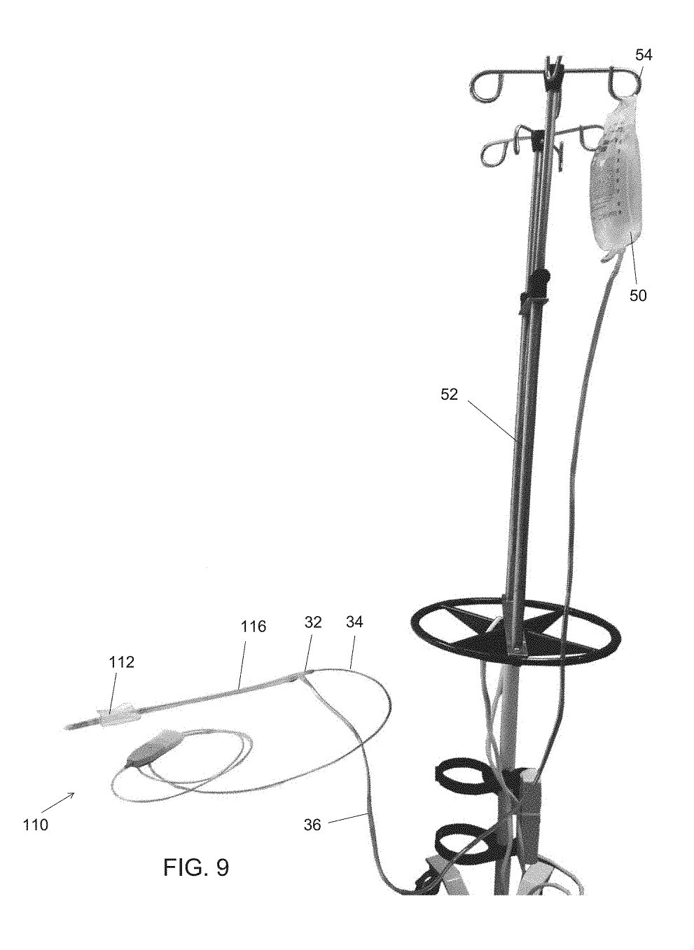

Alternatively, it has been noted that it may be advantageous to provide fluid based on the desired pressure to be achieved in the dilation and measurement balloon. For instance, in the apparatus 110 shown in FIG. 9, the fluid source connected to tube 36 may be a container 50 that is more similar to a source bag for intravenous (IV) fluids. The pressure in the dilation and measurement balloon 112 may be varied by adjusting the height at which the fluid source bag 50 is held above the dilation balloon 112, such as on the height adjustable pole 52, at a hook 54. The fluid source bag 50 will tend to fill the balloon 112 via the tube 36 from the fluid source bag 50 and via the catheter 116 connected to tube 36. It will be appreciated that, as the fluid source bag 50 is moved to a position that is raised or lowered relative to the balloon 112, the pressure in the balloon 112 will be increased or decreased, respectively. This could be accomplished by adjusting the height of the device from which the fluid source bag 50 is hanging, or the device may have multiple positions along its height at which the fluid source bag 50 may be connected, such as a plurality of hooks. Similarly, in such a pressure regulating configuration, the pressure may be decreased by lowering the fluid source bag 50 relative to the height of the dilation and measurement balloon 112. The pressure may be monitored and the fluid source bag 50 may be suspended, such as is shown on the portable, height adjustable pole 52, and moved to a height sufficient to fill the dilation balloon 112 and to generate a desired pressure level within the dilation balloon 112, such as the pressure levels discussed previously herein. It is believed that consistency in the pressure applied by the dilation and measurement balloon 112 will provide more stable progression of the desired dilation and effacement. Also, if the fluid source bag is vented, the apparatus may protect against or account for circumstances when the dilation balloon 112 may be subjected to movements that could cause a spike in pressures. The venting may permit fluid to be expressed back into the catheter 16 and tube 36, and ultimately into the fluid source bag 50, or there may be a different or additional provision for relieving excessive pressure, such as use of a relief valve 40.

It will be appreciated that the apparatus may be employed to provide a method of promoting and assessing dilation of a cervix with a catheter apparatus that is connected to an ambulatory system. For instance, an ambulatory system may include a sensor/controller for the dilation and measurement catheter apparatus that is attached to the patient. The attachment could be achieved by a removable strap or other suitable fastening device that will hold the controller in place. The sensor/controller may be controlled directly by the patient by input to a screen, keypad or other interface provided on the sensor/controller. In another embodiment the sensor/controller may be very small and connected wirelessly to a larger instrument having direct input controls for the patient to monitor and remotely control the sensor/controller. In another embodiment, the sensor/controller of the ambulatory system on the patient, or the smaller sensor/controller may be controlled and report information to a mobile application that may both control the process and report the progress in real time, such as on a cellular device. This process and the software to control and report the process with this apparatus may be shared via the internet with a health care professional and/or provider and the patient, allowing the patient to be able to be mobile and, depending on the circumstances, to be dismissed to go home and to continue the process of CRIB and the measurements to assess progress toward readiness for labor. Thus, the apparatus may further comprise a controller that may be carried and operated by the patient. Such apparatus may be part of an ambulatory system that provides enhanced mobility to the user. The apparatus also may further communicate via the internet to provide information to third parties.

It will be appreciated that the construction and operation of such apparatus may vary and may be suitable for other uses and/or may include additional or alternative desirable features.

From the above description, it will be appreciated that this disclosure has presented examples of an apparatus for promoting and assessing dilation of a cervix during induction of labor. Each of the examples provides an apparatus that includes a catheter having at least one lumen, at least one dilation balloon connected to and in fluid communication with the at least one lumen of the catheter, the at least one dilation balloon extending along and encircling a length of the catheter, and the at least one dilation balloon being configured to be filled with fluid to expand sufficiently to promote dilation of the cervix. Each apparatus further includes a plurality of sensors connected to the at least one lumen of the catheter and being spaced apart along the length of the catheter encircled by the at least one dilation balloon, and the plurality of sensors being configured to measure the diameter of the at least one dilation balloon along the length of the catheter encircled by the at least one dilation balloon. It will be appreciated that the apparatus also may include the further components of a CRIB system for filling a dilation balloon, or alternative filling structures, and of the EndoFLIP® System for communicating with the sensors to obtain and process the measurements of the diameter of the dilation balloon.

From the above disclosure, it will be appreciated that apparatus for promoting and assessing dilation of a cervix during induction of labor that are constructed in accordance with the present disclosure may be provided in various configurations. Any variety of suitable materials of construction, configurations, shapes and sizes for the components and methods of connecting the components may be utilized to meet the particular needs and requirements of an end user. It will be apparent to those skilled in the art that various modifications can be made in the design and construction of such apparatus and in the implementation of the method of use, without departing from the scope of the claimed subject matter, and that the claims are not limited to the preferred embodiments illustrated herein. Indeed, apparatus constructed in accordance with this disclosure may include a number of alternative structural aspects that may exhibit one or more of the above-referenced potential advantages over conventional medical devices, depending upon the specific design chosen. It also will be appreciated that the example embodiments are shown in simplified form, so as to focus on the key components and to avoid including structures that are not necessary to the disclosure and that would overly complicate the drawings.

1. An apparatus for promoting and assessing dilation of a cervix during induction of labor comprising:
a catheter having at least one lumen;
at least one dilation balloon connected to and in fluid communication with the at least one lumen of the catheter;
the at least one dilation balloon extending along and encircling a length of the catheter;
the at least one dilation balloon being configured to be filled with fluid to expand sufficiently to promote dilation of the cervix;
a plurality of sensors connected to the at least one lumen of the catheter and being spaced apart along the length of the catheter encircled by the at least one dilation balloon; and
the plurality of sensors being configured to measure the diameter of the at least one dilation balloon along the length of the catheter encircled by the at least one dilation balloon.

2. The apparatus of claim 1 wherein the sensors provide periodic respective diameter measurements of the at least one dilation balloon.

3. The apparatus of claim 2 wherein the sensors are electrodes that measure voltage.

4. The apparatus of claim 3 wherein the electrodes measure the diameter of the at least one dilation balloon by utilizing impedance planimetry.

5. The apparatus of claim 1 wherein the catheter further comprises a bullet-shaped distal tip.

6. The apparatus of claim 1 wherein the catheter further comprises a second lumen in fluid communication with a second balloon positioned along the catheter and being located adjacent a distal end of the at least one dilation balloon.

7. The apparatus of claim 1 further comprising a fluid source connected to the catheter having at least one lumen, with the fluid source being height adjustable with respect to the at least one dilation balloon, wherein the fluid source is used to fill the at least one dilation balloon and the pressure in the dilation balloon is adjusted based on the height of the fluid source relative to the at least one dilation balloon.

8. The apparatus of claim 1 wherein the catheter further comprises a relief valve in fluid communication with the catheter and at least one dilation balloon.

9. A method of promoting and assessing dilation of a cervix with a catheter apparatus having at least one lumen connected to at least one dilation balloon that encircles and extends along a length of the catheter and a plurality of sensors being spaced apart and providing a diameter measurement of the at least one dilation balloon along the length of the catheter encircled by the dilation balloon, the method comprising;
positioning the at least one dilation balloon within the cervix;
filling the at least one dilation balloon with a fluid to expand sufficiently to promote dilation of the cervix;
using the sensors to measure the diameter of the at least one dilation balloon along the length of the catheter encircled by the at least one dilation balloon; and
periodically assessing the measurements of the diameter of the at least one dilation balloon along the length of the catheter encircled by the at least one dilation balloon to assess dilation of the cervix.

10. The method of claim 9 wherein the sensors are electrodes that measure voltage.

11. The method of claim 10 wherein the electrodes measure the diameter of the at least one dilation balloon by utilizing impedance planimetry.

12. The method of claim 9 wherein the catheter apparatus further comprises a bullet-shaped distal tip.

13. The method of claim 9 wherein the catheter apparatus further comprises a second lumen connected to a second balloon positioned along the catheter and being located adjacent to a distal end of the at least one dilation balloon.

14. The method of claim 13 further comprising filling the second balloon with fluid.

15. The method of claim 9 wherein a fluid source is connected to the catheter having at least one lumen, with the fluid source being height adjustable with respect to the at least one dilation balloon, wherein the fluid source is used to fill the at least one dilation balloon and the pressure in the dilation balloon is adjusted by raising or lowering the height of the fluid source relative to the at least one dilation balloon.

16. The method of claim 9 wherein the apparatus further comprises a relief valve and pressure in the at least one dilation balloon is the relieved via the relief valve.
